(19) United States
(12) Patent Application Publication
(10) Pub. No.: US 2017/0280977 A1
(43) Pub. Date: Oct. 5, 2017

(54) CAPSULE ENDOSCOPE AND CAPSULE ENDOSCOPE SYSTEM

(71) Applicant: OLYMPUS CORPORATION, Tokyo (JP)

(72) Inventor: Takuto IKAI, Tokyo (JP)

(73) Assignee: OLYMPUS CORPORATION, Tokyo (JP)

(21) Appl. No.: 15/623,557

(22) Filed: Jun. 15, 2017

Related U.S. Application Data
(63) Continuation of application No. PCT/JP2015/075563, filed on Sep. 9, 2015.

(30) Foreign Application Priority Data
Dec. 19, 2014 (JP) 2014-257781

(51) Int. Cl. A61B 1/00; A61B 1/04

(52) U.S. Cl. CPC A61B 1/00158; A61B 1/041

(57) ABSTRACT

A capsule endoscope includes: a function-performing device configured to perform a function; a power source configured to supply power; a first inductor including a cored coil, the first inductor being configured to boost a voltage of the power and output a first voltage; a second inductor including an air-cored coil, the second inductor being configured to boost a voltage of the power and output a second voltage; a detector configured to detect a magnetic field applied from outside; and a controller configured to perform control to supply first power according to the first voltage to the function-performing device, when the magnetic field detected by the detector has a magnitude less than a threshold, and to supply second power according to the second voltage to the function-performing device, when the magnetic field detected by the detector has a magnitude not less than a threshold.

### CROSS REFERENCES TO RELATED APPLICATIONS

This application is a continuation of PCT international application Ser. No. PCT/JP2015/075563 filed on Sep. 9, 2015 which designates the United States, incorporated herein by reference, and which claims the benefit of priority from Japanese Patent Applications No. 2014-257781, filed on Dec. 19, 2014, incorporated herein by reference.

### BACKGROUND

### 1. Technical Field

The disclosure relates to a capsule endoscope and a capsule endoscope system which are configured to introduce the capsule endoscope into a subject to acquire an in-vivo image in the subject.

### 2. Related Art

Capsule endoscopes are known to be orally introduced into subjects to capture inside the subjects, and wirelessly transmit acquired image information to external devices positioned outside the subjects. Such a capsule endoscope includes a voltage booster circuit using a core coil as an inductor. The voltage booster circuit boots a voltage of power supplied from a power supply to voltages suitable for function-performing devices, such as an illumination unit and an imaging unit, disposed in the capsule endoscope, to supply the power to the function-performing devices (see JP 2008-119056 A).

### SUMMARY

In some embodiments, a capsule endoscope includes: a function-performing device configured to perform a function; a power source configured to supply power; a first inductor including a cored coil, the first inductor being configured to boost a voltage of the power and output a first voltage; a second inductor including an air-cored coil, the second inductor being configured to boost a voltage of the power and output a second voltage; a detector configured to detect a magnetic field applied from outside; and a controller configured to perform control to supply first power according to the first voltage to the function-performing device, when the magnetic field detected by the detector has a magnitude less than a threshold, and to supply second power according to the second voltage to the function-performing device, when the magnetic field detected by the detector has a magnitude not less than a threshold.

In some embodiments, a capsule endoscope system includes: a magnetic field generator configured to generate a magnetic field acting on a capsule endoscope to be introduced into a subject; an operation input device configured to input instruction information for changing the magnetic field to guide the capsule endoscope; and a transmitter configured to wirelessly transmit the instruction information to the capsule endoscope. The capsule endoscope includes: a function-performing device configured to perform a function; a permanent magnet; a power source configured to supply power; a first inductor including a cored coil, the first inductor being configured to boost a voltage of the power and output a first voltage; a second inductor including an air-cored coil, the second inductor being configured to boost a voltage of the power and output a second voltage; a receiver configured to receive the instruction information transmitted from the transmitter; and a controller configured to perform control to supply first power according to the first voltage to the function-performing device, when the receiver does not receive the instruction information, and to supply second power according to the second voltage to the function-performing device, when the receiver receives the instruction information.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an exemplary configuration of a capsule endoscope system according to a first embodiment of the disclosure;

FIG. 2 is a schematic diagram illustrating an exemplary internal structure of a capsule endoscope according to the first embodiment of the disclosure;

FIG. 3 is a flowchart illustrating an outline of a process performed by the capsule endoscope according to the first embodiment of the disclosure;

FIG. 4 is a diagram illustrating an exemplary configuration of a capsule endoscope system according to a second embodiment of the disclosure;

FIG. 5 is a schematic diagram illustrating an exemplary internal structure of a capsule endoscope according to the second embodiment of the disclosure; and

FIG. 6 is a flowchart illustrating an outline of a process performed by the capsule endoscope according to the second embodiment of the disclosure.

### DETAILED DESCRIPTION

A capsule endoscope system according to embodiments of the disclosure will be described below with reference to the drawings. Note that, in the following description, a capsule endoscope orally introduced into a subject to perform capturing while flowing in a liquid stored in a stomach of the subject is exemplified, but the disclosure is not limited to these embodiments. That is, for example, the disclosure can employ various capsule endoscopes such as a capsule endoscope capturing inside a digestive tract while being moved from an esophagus to an anus of a subject by peristaltic motion, or a capsule endoscope introduced from an anus with isotonic solution. Furthermore, in the following description, the drawings merely schematically illustrate shapes, sizes, and positional relationships to the extent that the contents of the disclosure can be understood. Accordingly, the disclosure is not limited only to the shapes, sizes, and positional relationships exemplified in the drawings. Note that, in the drawings, the same portions are denoted by the same reference signs.

### First embodiment

Configuration of Capsule Endoscope System

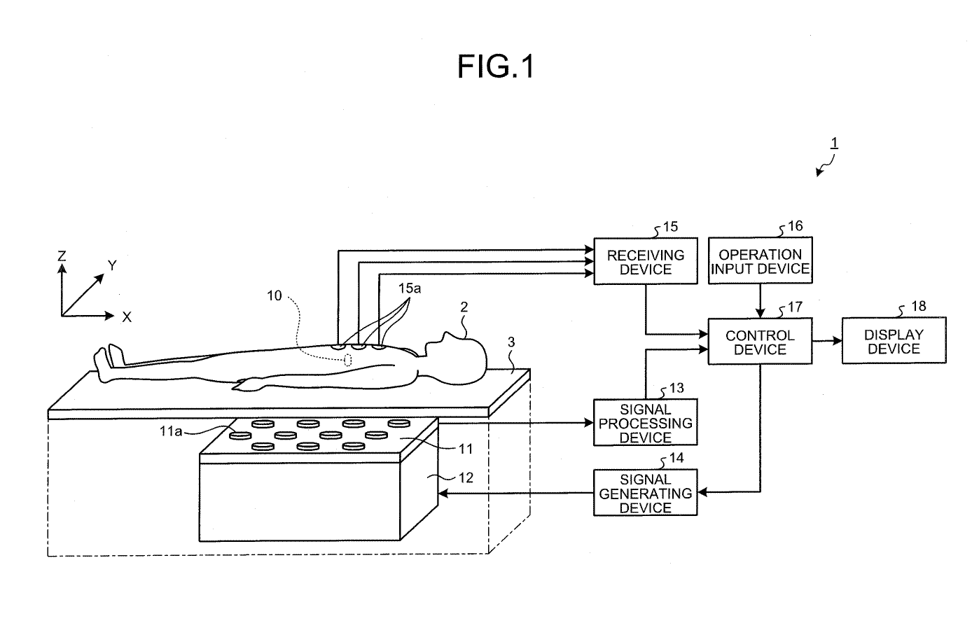

FIG. 1 is a diagram illustrating an exemplary configuration of a capsule endoscope system according to a first embodiment of the disclosure. A capsule endoscope system 1 illustrated in FIG. 1 includes a capsule endoscope 10 configured to be introduced into a digestive tract of a subject 2 and wirelessly transmit an image signal (image information) acquired by capturing inside the subject 2, a position detection device 11 configured to detect a position of the capsule endoscope 10 through a plurality of sense coils 11a configured to be provided under a bed 3 on which the subject 2 is laid, a magnetic field generating device 12 configured to generate a magnetic field acting on the capsule endoscope 10, a signal processing device 13 configured to process a signal output from the position detection device 11, a signal generating device 14 configured to generate a signal for operating the magnetic field generating device 12, a receiving device 15 configured to receive image signals wirelessly transmitted from the capsule endoscope 10, through a plurality of receiving antennas 15a, respectively, an operation input device 16 configured to perform guidance operation of the capsule endoscope 10, a control device 17 configured to perform a process to display an image captured in the subject 2 (herein after, referred to as “in-vivo image”), on the basis of the image signals received by the receiving device 15, and a display device 18 configured to display an in-vivo image or other information. Note that the bed 3 is disposed so that an upper surface (a mounting surface for the subject 2) is parallel to a horizontal plane (plane orthogonal to the gravity direction). In the following description, a longitudinal direction of the bed 3 is defined as an X direction, a transverse direction of the bed 3 is defined as a Y direction, and a vertical direction (gravity direction) thereof is defined as a Z direction. Furthermore, in the first embodiment, the magnetic field generating device 12 functions as a magnetic field generator.

Configuration of Capsule Endoscope

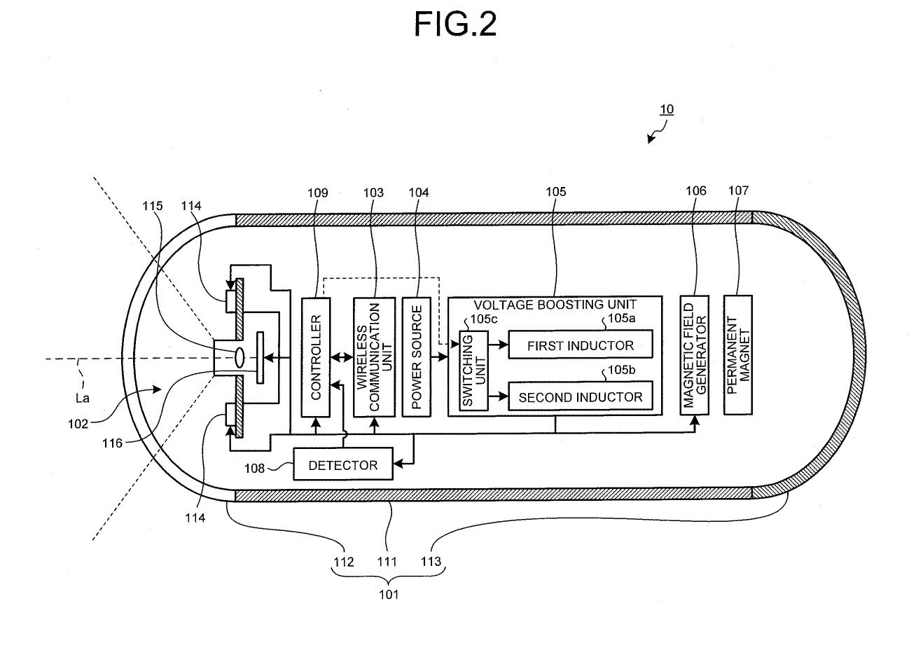

Next, a configuration of the capsule endoscope will be described. FIG. 2 is a schematic diagram illustrating an exemplary internal structure of the capsule endoscope 10. The capsule endoscope 10 illustrated in FIG. 2 includes a capsule-shaped casing 101 configured to have an outer casing formed to have a size sufficient to be readily introduced into an organ of the subject 2, an imaging unit 102 configured to capture the subject 2 and generating an image signal, a wireless communication unit 103 configured to wirelessly transmit the image signal generated by the imaging unit 102 to the outside, a power source 104 configured to supply power to each component unit of the capsule endoscope 10, a voltage boosting unit 105 configured to boost voltage supplied from the power source 104 to a predetermined voltage, a magnetic field generator 106 configured to generate an alternating magnetic field for detecting position of the capsule endoscope 10, a permanent magnet 107 configured to allow magnetic induction by the magnetic field generating device 12, a detector 108 configured to detect the magnetic field generated by the magnetic field generating device 12, and a controller 109 configured to control each component unit of the capsule endoscope 10.

The capsule-shaped casing 101 is an outer casing formed to have a size sufficient to be introduced into an organ of the subject 2, and is achieved by closing both opening ends of a cylindrical casing 111 by dome-shaped casings 112 and 113. The dome-shaped casing 112 is a dome-shaped optical members transparent to light in a predetermined wavelength band, such as visible light. Furthermore, the cylindrical casing 111 and the dome-shaped casing 113 are colored casings substantially opaque to visible light. The capsule-shaped casing 101 formed by the cylindrical casing 111 and the dome-shaped casings 112 and 113 liquid-tightly encloses the imaging unit 102, the wireless communication unit 103, the power source 104, the voltage boosting unit 105, the magnetic field generator 106, the permanent magnet 107, the detector 108, and the controller 109, as illustrated in FIG. 2.

The imaging unit 102 includes an illumination unit 114 such as a light emitting diode (LED), an optical system 115 such as a condenser lens, and an image sensor 116 such as a complementary metal oxide semiconductor (CMOS) or a charge coupled device (CCD).

Under the control of the controller 109, the illumination unit 114 emits illumination light, such white light, to a field of view of the image sensor 116, and illuminates an object in the field of view through the dome-shaped casing 112.

The optical system 115 focuses light reflected from the field of view to a capturing surface of the image sensor 116 to form an image of the object. The optical system 115 includes at least one or more lenses.

The image sensor 116 receives light reflected from the field of view and focused on the capturing surface, and photoelectrically converts an optical signal of the received light to generate an image signal representing the image of the object in the field of view, that is, an in-vivo image of the subject 2.

Note that, in the present embodiment, only one imaging unit 102 is provided in the capsule endoscope 10, but the imaging unit 102 may be provided also in the dome-shaped casing 113 to capture portions in front and back of the capsule endoscope in a major axis La. In this configuration, the dome-shaped casing 113 is also formed of an optical member transparent to light in a predetermined wavelength band, such as visible light. Furthermore, in this configuration, the two imaging units 102 are disposed to respectively have optical axes substantially parallel to or substantially coincide with the major axis La as a center axis of the capsule-shaped casing 101 in a longitudinal direction, and respectively have fields of view facing opposite to each other.

The wireless communication unit 103 wirelessly transmits image signals generated by the imaging unit 102 to the outside in sequence, through an antenna not illustrated. Specifically, the wireless communication unit 103 acquires an image signal generated by the imaging unit 102 from the controller 109, and performs signal processing such as modulation on the image signal to generate a wireless signal. The wireless communication unit 103 transmits the wireless signal to the receiving device 15 provided outside the subject 2.

The power source 104 is a power storage unit, such as a button battery or a capacitor, and has a magnetic switch, an optical switch, or a switch portion (not illustrated) switched by a command from the controller 109. The power source 104, for example, receives a high-frequency signal having a specific pattern, as a command for switching the switch portion, applied from outside through the wireless communication unit 103, switches between on and off states of power supply through control by the controller 109 based on the high-frequency signal, and supplies power from the power storage unit to the voltage boosting unit 105, during the on state. Furthermore, during the off state, the power source 104 stops supplying the power to the voltage boosting unit 105.

The voltage boosting unit 105 boosts a voltage of the power supplied from the power source 104 to a predetermined voltage. The voltage boosting unit 105 includes a first inductor 105a, a second inductor 105b, and a switching unit 105c.

The first inductor 105a includes a cored coil having a core to boost the power supplied from the power source 104, and appropriately supplies the boosted power to each function-performing device of the capsule endoscope 10 (the imaging unit 102, the wireless communication unit 103, the magnetic field generator 106, the detector 108, and the controller 109). Specifically, the first inductor 105a boosts the voltage of the power supplied from the power source 104 through the switching unit 105c, and supplies the boosted voltage to each function-performing device of the capsule endoscope 10.

The second inductor 105b includes an air-cored coil to boost the power supplied from the power source 104, and appropriately supplies the boosted power to each function-performing device of the capsule endoscope 10 (the imaging unit 102, the wireless communication unit 103, the magnetic field generator 106, the detector 108, and the controller 109). Specifically, the second inductor 105b boosts the voltage of the power supplied from the power source 104 through the switching unit 105c, and supplies the boosted voltage to each function-performing device of the capsule endoscope 10.

Under the control of the controller 109, the switching unit 105c switches a supply destination of the power supplied from the power source 104 to either the first inductor 105a or the second inductor 105b. The switching unit 105c includes a switch or the like.

The magnetic field generator 106 includes a transmission coil configured to partially constitute a resonance circuit, and generate a magnetic field when electric current flows, and a capacitor configured to form the resonance circuit with the transmission coil, and the magnetic field generator 106 receives power supply from the voltage boosting unit 105 to generate the alternating magnetic field having a predetermined frequency.

The permanent magnet 107 is fixedly disposed in the capsule-shaped casing 101 to have a magnetization direction inclined relative to the major axis La. In the first embodiment, the permanent magnet 107 is disposed to have a magnetization direction orthogonal to the major axis La. The permanent magnet 107 works following a magnetic field applied from outside, and thus, achieves magnetic induction for the capsule endoscope 10, generated by a magnetic field generating device 12, which is described later.

The detector 108 determines whether the magnetic field is applied from outside. Specifically, the detector 108 detects the magnetic field generated by the magnetic field generating device 12, and outputs a result of the detection to the controller 109. The detector 108 includes for example a magnetic sensor. Note that, in the first embodiment, the detector 108 functions as a determination unit.

The controller 109 includes a central processing unit (CPU) or the like, controls operations of the imaging unit 102, the wireless communication unit 103, and the voltage boosting unit 105, and controls input/output of signals between these component units. Specifically, whenever the image sensor 116 generates an image signal, the controller 109 acquires this image signal to perform predetermined signal processing, and further controls the wireless communication unit 103 to wirelessly transmit this image signal to the outside in time series.

Furthermore, the controller 109 causes any one of the first inductor 105a and the second inductor 105b to supply the power from the power source 104 to the function-performing devices. Specifically, on the basis of a result of the detection made by the detector 108, the controller 109 controls the switching unit 105c to perform switching control between supplying the power supplied from the power source 104 to each function-performing device through the first inductor 105a, and supplying the power supplied from the power source 104 to each function-performing device through the second inductor 105b. More specifically, when a result of the detection made by the detector 108 is less than a predetermined threshold, the controller 109 performs switching control to supply the power from the power source 104 to each function-performing device through the first inductor 105a, and when a result of the detection made by the detector 108 is not less than the predetermined threshold, the controller 109 performs switching control to supply the power from the power source 104 to each function-performing device through the second inductor 105b.

Process by Capsule Endoscope

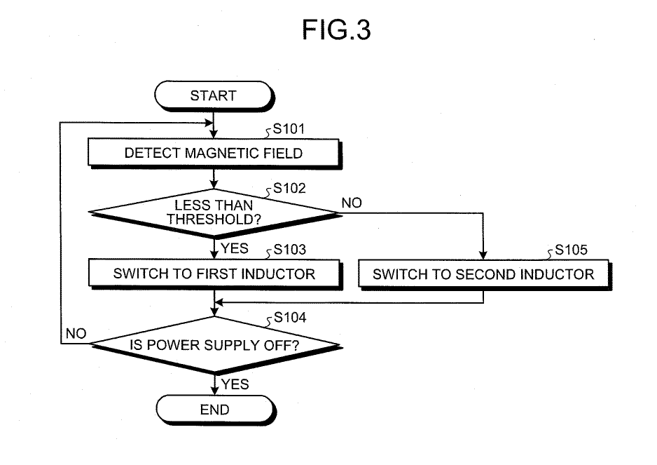

A process performed by the capsule endoscope 10 having the configuration described above will be described. FIG. 3 is a flowchart illustrating an outline of the process performed by the capsule endoscope 10.

As illustrated in FIG. 3, first, the detector 108 detects an intensity of a magnetic field (step S101), and when a result of the detection made by the detector 108 is less than a predetermined threshold (step S102: Yes), the controller 109 controls the switching unit 105c to switch the supply destination of the power to supply the power from the power source 104 to the first inductor 105a (step S103).

Then, for example, when a power supply voltage is not more than a predetermined value which is less than the threshold, and power supply from the power source 104 is in an off state (step S104: Yes), the capsule endoscope 10 finishes this process. On the other hand, when the power supply from the power source 104 is not in the off state (step S104: No), the process returns to step S101.

In step S102, when a result of the detection made by the detector 108 is not less than the threshold (step S102: No), the controller 109 controls the switching unit 105c to switch the supply destination of the power to supply the power from the power source 104 to the second inductor 105b (step S105). After step S105, the capsule endoscope 10 proceeds to step S104.

According to the first embodiment described above, the controller 109 switches between the first inductor 105a and the second inductor 105b, on the basis of a result of the detection made by the detector 108, so that even if the magnetic field for magnetic induction is applied from outside, the imaging unit 102 and the like can be prevented from being shut down.

Furthermore, according to the first embodiment, when a result of the detection made by the detector 108 is less than the predetermined threshold, the controller 109 performs control to supply power to the imaging unit 102 and the wireless communication unit 103 through the first inductor 105a, and when a result of the detection made by the detector 108 is not less than the predetermined threshold, the controller 109 performs control to supply power to the imaging unit 102 and the wireless communication unit 103 through the second inductor 105b, and thus, the imaging unit 102 and the like can be prevented from being shut down.

First Modification of First Embodiment

Note that, in the first embodiment, the detector 108 includes the magnetic sensor, but the detector 108 may include an acceleration sensor detecting acceleration generated in the capsule endoscope 10, instead of the magnetic sensor. In this configuration, when a result of detection made by the acceleration sensor is less than a predetermined threshold, the controller 109 controls the switching unit 105c to switch the supply destination of the power to supply the power from the power source 104 to the first inductor 105a, and when a result of detection made by the acceleration sensor is not less than the predetermined threshold, the controller 109 controls the switching unit 105c to switch the supply destination of the power to supply the power from the power source 104 to the second inductor 105b, and thus the imaging unit 102 and the like can be prevented from being shut down.

Second Modification of First Embodiment

Furthermore, in the first embodiment, a dimming sensor may be employed instead of the magnetic sensor. In this configuration, when a result of detection made by the dimming sensor is less than a predetermined threshold, the controller 109 controls the switching unit 105c to switch the supply destination of the power to supply the power from the power source 104 to the first inductor 105a, and when a result of detection made by the dimming sensor is not less than the predetermined threshold, the controller 109 controls the switching unit 105c to switch the supply destination of the power to supply the power from the power source 104 to the second inductor 105b, and thus the imaging unit 102 and the like can be prevented from being shut down.

### Second Embodiment

Next, a second embodiment of the disclosure will be described. A capsule endoscope according to the second embodiment is different from the capsule endoscope according to the first embodiment described above, in configuration, and further in process to be performed. Specifically, the capsule endoscope according to the second embodiment switches between the first inductor and the second inductor, according to the contents of an operation signal from the operation input device 16. Thus, in the following description, after description of a configuration of a capsule endoscope system according to the second embodiment, a process performed by the capsule endoscope will be described. Note that the same configurations as those of the capsule endoscope system 1 according to the first embodiment described above are denoted by the same reference signs, and description thereof will be omitted.

Configuration of Capsule Endoscope System

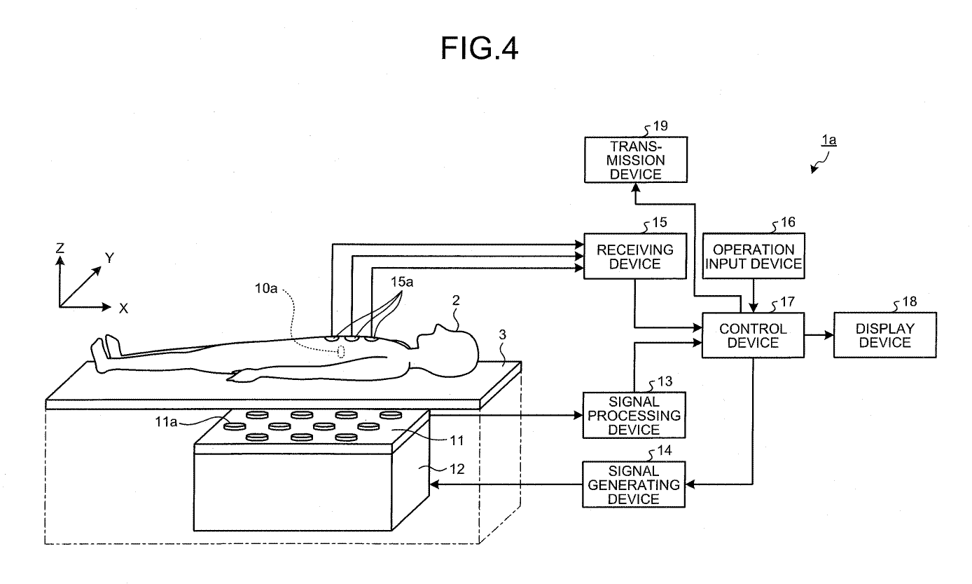

FIG. 4 is a diagram illustrating an exemplary configuration of a capsule endoscope system according to the second embodiment. A capsule endoscope system 1a illustrated in FIG. 4 includes a capsule endoscope 10a, instead of the capsule endoscope 10 of the capsule endoscope system 1 according to the first embodiment described above. The capsule endoscope system la further includes a transmission device 19 configured to transmit an operation signal input from the operation input device 16 to the capsule endoscope 10a.

Under the control of the control device 17, the transmission device 19 transmits an operation signal input from the operation input device 16 to the capsule endoscope 10a. The transmission device 19 transmits an inductor switching instruction signal corresponding to instruction input made from the operation input device 16. The transmission device 19 includes a modulation circuit, an antenna, and the like. The modulation circuit modulates an operation signal, and the antenna transmits the operation signal modulated by the modulation circuit to the capsule endoscope 10a.

Configuration of Capsule Endoscope

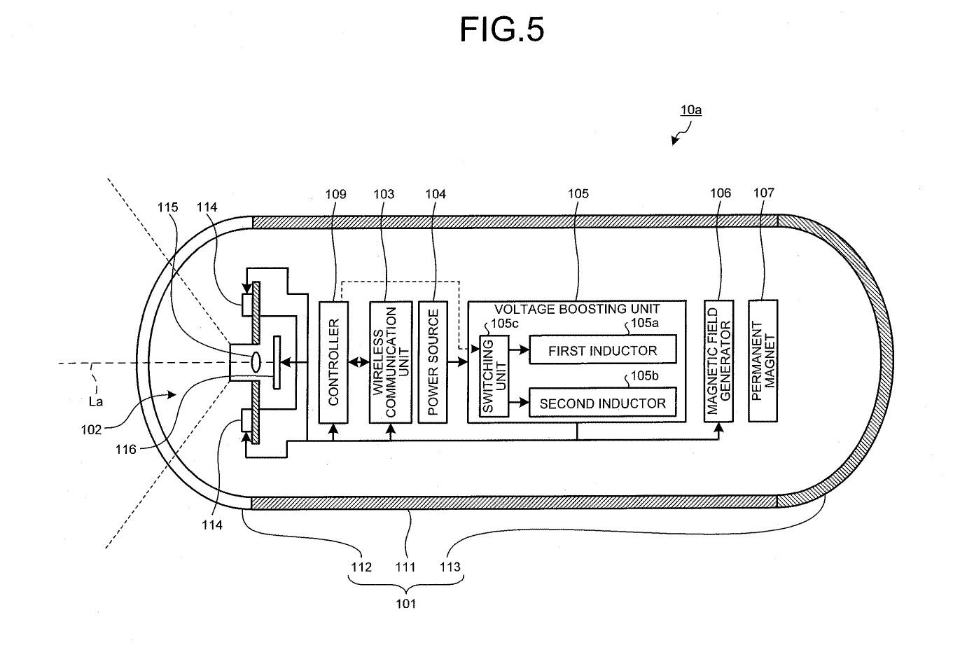

Next, a configuration of the capsule endoscope 10a will be described. FIG. 5 is a schematic diagram illustrating an exemplary internal structure of the capsule endoscope 10a. In the capsule endoscope 10a of FIG. 5, the detector 108 is omitted from the configuration of the capsule endoscope 10 according to the first embodiment described above. The other configuration is similar to that of the capsule endoscope 10 according to the first embodiment described above.

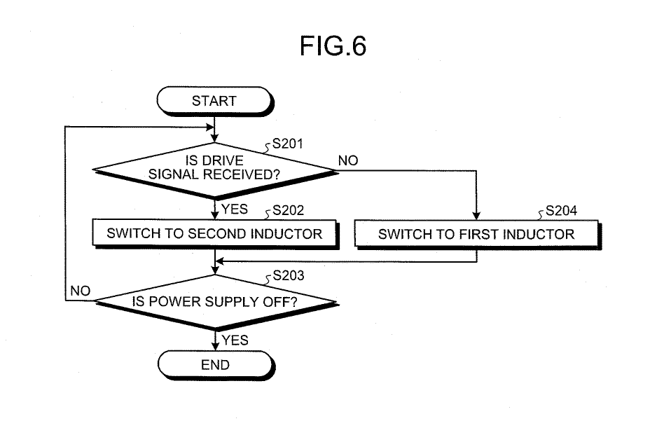

Process by Capsule Endoscope Next, a process performed by the capsule endoscope 10a will be described. FIG. 6 is a flowchart illustrating an outline of the process performed by the capsule endoscope 10a.

At the start of the process of FIG. 6, power from the power source 104 is preset to be supplied to the first inductor 105a. When, in order to move the capsule endoscope 10a, instruction input is transmitted from the operation input device 16 to drive the magnetic field generating device 12 to generate a magnetic field, the inductor switching instruction signal is transmitted from the transmission device 19, corresponding to the instruction input. At the same time as the magnetic field is generated from the magnetic field generating device 12, corresponding to the instruction input, the capsule endoscope 10a receives the inductor switching instruction signal from the transmission device 19, through the wireless communication unit 103 (step S201: Yes), and the controller 109 controls the switching unit 105c to switch the supply destination of the power to supply the power from the power source 104 to the second inductor 105b (step S202).

Then, for example, when a power supply voltage is not more than a predetermined value which is less than the threshold, owing to the magnetic field applied from outside, and power supply from the power source 104 is in the off state (step S203: Yes), the capsule endoscope 10a finishes this process. On the other hand, when the power supply from the power source 104 is not in the off state (step S203: No), the process returns to step S201.

Meanwhile, when a drive signal driving the magnetic field generating device 12 is not received from the transmission device 19 through the wireless communication unit 103 (step S201: No), the controller 109 controls the switching unit 105c to maintain a state in which the power from the power source 104 is set to be supplied to the first inductor 105a, or when the power from the power source 104 is set to be supplied to the second inductor 105b, the controller 109 controls the switching unit 105c to switch the supply destination of the power to supply the power from the power source 104 to the first inductor 105a (step S204). After step S204, the process proceeds to step S203.

According to the second embodiment described above, on the basis of a reception result of the inductor switching instruction signal from the transmission device 19, the controller 109 switches between the first inductor 105a and the second inductor 105b, to supply the power from the power source 104 to the imaging unit 102 or the like, and the imaging unit 102 and the like can be prevented from being shut down.

According to some embodiments, even if a magnetic field for magnetic induction is applied from outside, the function-performing devices are effectively prevented from being shut down.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A capsule endoscope comprising:
a function-performing device configured to perform a function;
a power source configured to supply power;
a first inductor including a cored coil, the first inductor being configured to boost a voltage of the power and output a first voltage;
a second inductor including an air-cored coil, the second inductor being configured to boost a voltage of the power and output a second voltage;
a detector configured to detect a magnetic field applied from outside; and
a controller configured to perform control to supply first power according to the first voltage to the function-performing device, when the magnetic field detected by the detector has a magnitude less than a threshold, and to supply second power according to the second voltage to the function-performing device, when the magnetic field detected by the detector has a magnitude not less than a threshold.

2. A capsule endoscope system comprising:
a magnetic field generator configured to generate a magnetic field acting on a capsule endoscope to be introduced into a subject;
an operation input device configured to input instruction information for changing the magnetic field to guide the capsule endoscope; and
a transmitter configured to wirelessly transmit the instruction information to the capsule endoscope, wherein
the capsule endoscope including:
a function-performing device configured to perform a function;
a permanent magnet;
a power source configured to supply power;
a first inductor including a cored coil, the first inductor being configured to boost a voltage of the power and output a first voltage;
a second inductor including an air-cored coil, the second inductor being configured to boost a voltage of the power and output a second voltage;
a receiver configured to receive the instruction information transmitted from the transmitter; and
a controller configured to perform control to supply first power according to the first voltage to the function-performing device, when the receiver does not receive the instruction information, and to supply second power according to the second voltage to the function-performing device, when the receiver receives the instruction information.
